SEQUENCE LISTING (8 sequences)

SEQ ID NO: 1 - DNA, 3500 bp, Clostridium histolyticum
  source (1)..(3500)
  gene (301)..(3366)
  CDS (301)..(3366)
  sig_peptide (301)..(420)<s300> GenBank / D299812003-01-30
aactcctcccgttttaaatagaatctttataaatttattttatcctaatattctcttatatacttaattaaatattaataaaaaattaatgaacaggtatatcttaacaaaaattaaacaaaaattaaacaaatatataacaaatattaataaataatgttgacactactaaaaaatggcgttatactttaataaaaggcttatataattcctcaatacaaatattcagataattatgaaaagagcataaatgaaggaattatgaattttttaaaaattattttaaatagggggaagactatgaaaaggaaatgtttatctaaaaggcttatgttagctataacaatggctacaatatttacagtgaacagtacattaccaatttatgcagctgtagataaaaataatgcaacagcagctgtacaaaatgaaagtaagaggtatacagtatcatatttaaagactttaaattattatgacttagtagatttgcttgttaagactgaaattgagaatttaccagacctttttcagtatagttcagatgcaaaagagttctatggaaataaaactcgtatgagctttatcatggatgaaattggtagaagggcacctcagtatacagagatagatcataaaggtattcctactttagtagaagttgtaagagctggattttacttaggattccataacaaggaattgaatgaaataaacaagaggtcttttaaagaaagggtaataccttctatattagcaattcaaaaaaatcctaattttaaactaggtactgaagttcaagataaaatagtatctgcaacaggacttttagctggtaatgaaacagcgcctccagaagttgtaaataattttacaccaatacttcaagactgtataaagaatatagacagatacgctcttgatgatttaaagtcaaaagcattatttaatgttttagctgcacctacctatgatataactgagtatttaagagctactaaagaaaaaccagaaaacactccttggtatggtaaaatagatgggtttataaatgaacttaaaaagttagctctttatggaaaaataaatgataataactcttggataatagataacggtatatatcatatagcacctttagggaagttacatagcaataataaaataggaatagaaactttaacagaggttatgaaagtttatccttatttaagtatgcaacatttacaatcagcagatcaaattaagcgtcattatgattcaaaagatgctgaaggaaacaaaatacctttagataagtttaaaaaggaaggaaaagaaaaatactgtccaaaaacttatacatttgatgatggaaaagtaataataaaagctggtgctagagtagaagaagaaaaagttaaaagactatactgggcatcaaaggaagttaactctcaattctttagagtatacggaatagacaaaccattagaagaaggtaatccagatgatatattaacaatggttatctacaacagtcccgaagaatataaactcaatagtgttctatacggatatgatactaataatggtggtatgtatatagagccagaaggaactttcttcacctatgaaagagaagctcaagaaagcacatacacattagaagaattatttagacatgaatatacacattatttgcaaggaagatatgcagttccaggacaatggggaagaacaaaactttatgacaatgatagattaacttggtatgaagaaggtggagcagaattatttgcaggttctactagaacttctggaatattaccaagaaagagtatagtatcaaatattcataatacaacaagaaataatagatataagctttcagacactgtacattctaaatatggtgctagttttgaattctataattatgcatgtatgtttatggattatatgtataataaagatatgggtatattaaataaactaaatgatcttgcaaaaaataatgatgttgatggatatgataattatattagagatttaagttctaattatgctttaaatgataaatatcaagatcatatgcaggagcgcatagataattatgaaaatttaacagtgccttttgtagctgatgattatttagtaaggcatgcttataagaaccctaatgaaatttattctgaaatatctgaagtagcaaaattaaaggatgctaagagtgaagttaagaaatcacaatattttagtacctttactttgagaggtagttacacaggtggagcatctaaggggaaattagaagatcaaaaagcaatgaataagtttatagatgattcacttaagaaattagatacgtattcttggagtgggtataaaactttaactgcttatttcactaattataaagttgactcttcaaatagagttacttatgatgtagtattccacggatatttaccaaacgaaggtgattccaaaaattcattaccttatggcaagatcaatggaacttacaagggaacagagaaagaaaaaatcaaattctctagtgaaggctctttcgatccagatggtaaaatagtttcttatgaatgggatttcggagatggtaataagagtaatgaggaaaatccagagcattcatatgacaaggtaggaacttatacagtgaaattaaaagttactgatgacaagggagaatcttcagtatctactactactgcagaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggatagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataatattttattagttgaggtaactccatataatagcttagctatttcttatggagttactttttatatgtaataaaattttgacttaaattatgattttttgctataatggtttggaaattaatgatttataattt

SEQ ID NO: 2 - protein, 1021 aa, Clostridium histolyticum
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 3 - DNA, 357 bp, Clostridium histolyticum
ccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataa

SEQ ID NO: 4 - DNA, 4801 bp, Rattus norvegicus
  source (1)..(4801)
  5′UTR (1)..(388)
  CDS (389)..(3790)
  variation (2971)..(2979)
  variation (3250)..(3252)
  variation (3324)..(3326)
  3′UTR (3788)..(4801)
  polyA_signal (4784)..(4789)
  polyA_site (4801)..(4801)<s300> GenBank / U048421994-01-11
caaaaggagaagccatcagggaaggaatcctatctgcatatttcgtctttagccccatccctcattcccggtggggtttggaactttccatcaattctttccctgtctcatttctctttgagcctttgcctagctgtgcctgtcacagcgagaaatcagtcaccctccgccttccagcactcttaggctctgagaaatttggcatacgggtgtcaggtattaaaacagctaaataaaagatgccctggggctgaaggccagcgtggctggaagttctgggggtcagaagcctgactccgcctgctccaagctctagcaatttaagtcacccgggggttttttgttttggtttggtttggtttttcttgaccttagaaccaccgagaccatgctgttctcgctcaccttcctgtcggtgtttttaaagattactgtactcagtgtcacagcacagcagaccaggaactgtcagtcaggtcctctcgagagaagcgggactaccacgtatgccgccgccggtcctcccaggttcctgattttcttacaaggaaacagcatctttcggattaacacagatggaacaaatcaccagcaattggtggtggatgccggcgtctcagtggtcatggattttcattacaaggaagagagactctattgggtggatttagaaagacaacttttgcaaagagttttctttaatgggtcaggacaagagacagtgtgcaaggtggataagaatgtgtctgggctggccataaactggatagatggggagattctccggacggaccgatggaagggagtcatcacagtaacagatatgaacgggaacaattcccgtgttcttctgagttccttaaaacgtcctgcaaatatattagtggatccaacagagaggttgatattttggtcttcagtggtgactggcaaccttcacagagcagatctcgggggtatggatgtaaaaacactgctggaggcaccagagaggatatcagtgctgattctggatatcctggacaaaaggctcttctgggctcaggacggtagagaaggaagccacggttacattcactcctgtgactataacggtggctccatccatcatatcagacatcaagcacggcacgatttgcttactatggccattttcggtgacaagatcttatactcagcactgaaagagaaggcgatttggatagccgacaaacacactgggaagaatgtggttcgagttaacctcgatccagcctctgtgccgccaagagaactgagagtcgtgcacctacatgcacagcccgggacagagaaccgtgctcaggcctctgactccgaacgatgcaaacagagaagaggacagtgtctctacagtctctctgagcgagaccccaactcagactcgtcggcatgcgctgaaggctatacgttaagccgagaccggaagtactgcgaagatgtcaatgagtgtgccttgcagaatcacggctgtactcttgggtgtgaaaacatccctggatcctattactgcacatgccctacaggctttgttctgcttcctgatgggaaacgatgtcacgaacttgttgcctgtccaggcaacagatcagagtgtagccatgattgcatcctgacatcagatggtcctctgtgcatctgtccagcaggttcagtgctcggaaaagatgggaagacatgcactggttgttccttctccgataatggtggatgcagccagatctgccttcctctcagcctagcatcctgggaatgtgattgctttcctgggtacgacctacaattggaccgaaagagctgtgcagcttccatgggaccgcagccatttttactgtttgcaaattcccaggacatacgacacatgcattttgatggaacagactacaaaactctgctcagccggcagatgggaatggtttttgccttggattatgaccccgtggaaagcaagatatattttgcacagacagccctgaagtggatagagagggctaatctggatggctcccagcgagaaagacggatcacggaaggagtagacacgccagaaggtcttgccgtggactggattggccggagaatctactggacggacagtgggaagtctgtcattgaagggagtgatttgagcgggaagcatcatcaaataatcatcaaagagagcatctcaaggccacgaggaatagctgtgcatccaaaggccaggagactattctggacggacacggggatgtctccgcggattgaaagctcttcccttcaaggttctgaccggacgctgatagccagctctaatctactggaacccagtggaatcgcgattgactacttaacagacactttgtactggtgtgacaccaagctgtctgtgattgaaatggccgatctagatggttccaaacgccgcagacttacccagaacgatgtaggtcacccattctctctagctgtgtttgaggatcacgtgtggttctcggattgggctatcccatcggtaataagggtgaacaagaggactggtcaaaacagggtacgtctccgaggcagcatgctgaagccctcgtcactggttgtggtccacccattggcaaaaccaggtgcagacccctgcttacacaggaatggaggctgtgaacacatctgccaagagagcctgggcacggctcagtgtctgtgtcgggaaggattcgtgaaggccccagatgggaaaatgtgtctcactcggaaggatgatcagatactggccggtgacaatgctgatcttagtaaagaggtggcatcgttggacaactcccctaaggcttatgtaccagacgatgataggacagagtcctccacactagtggctgagatcatggtgtcagggctgaactatgaagatgactgcggccctggtgggtgtggcagccatgcccactgtatttcagagggagaggcagctgtgtgtcagtgtttgaaaggatttgctggcgatggaaacctgtgttctgatatagacgaatgtgagctgggtagctcagactgtcctcccacctcgtccaggtgcatcaacaccgaaggtggctatgtctgccaatgctcagaaggctacgagggagatgggatctactgtctcgacgttgatgagtgccagcaggggtcgcacggctgcagcgagaatgccacctgcaccaacacggagggaggctacaactgcacctgtgcaggctgcccatcagcacctggactgccttgccctgactctacctcaccctctctccttggaaaagatggctgccactgggtccgaaacagtaacacaggatgcccgccgtcgtacgatgggtactgcctcaatggtggcgtgtgcatgtatgttgaatccgtggaccgctacgtgtgcaactgtgtcattggctatattggagaacgatgtcagcaccgagacttacgttggtggaagctgcgccatgctgactacgggcagaggcacgacatcactgtggtgtctgtctgtgtggtggcgctggccctgctgctcctcttagggatgtgggggacttactactacaggactcggaagcagctatcagagagctcaaagaagccttccgaagagtcaagcagcaacgtgagcagtaacgggcctgacagcagcggggctggggtgtcttctggtccccaaccttggtttgtggtcctagaggaacaccaacagcccaagaatgggcgtctgcctgccgctggcacgaacggcgcagtagtagaggctggcctgtcttcctccctgtaactcgggccagtgcacctgacttcctggagacagaagccccgaatatatgagatgggcacagagcaaagctgctggattccaccatcaaatgacaaaggaccccaggaaatggaggggaacccccacttaccctcctacagggaatggcctctagctgtgtgggctgagaagaagctgcattctctccagtcagctaatggatcgagtcaacaaagggcctcagacctgccccagcaaacagagccagttctgtagaaactgggagcagacagaaggtaccgaaagtgaaatagcaaaccaggctgaagggtggtagagcggcagatctggtactcctgtctccacggctaatcactgctcagggtcctgaagataactgcatagctgcatagctgatagccgcgacttctgcttcttgcttcaagcagtcccgttgaagacgatcaaaagagaagtggagaaaaatcatcagaaaccgaagtcaagacggttcacgtgtgtaagctgtgtccttcctacccctggactgttgggctcttttccttgttgtctcagaagaaatgggttaaagcaggcgatcacatgctttgttgattgcacagtagatgatatgatctacatagatcttagctcactctcacggaaaggctggaacattatagatgctgcaagatacactgcaagtgtggcccctgctcataattttgccttctgaattgtgattagtgaaaataattgtaacttagagtccgatttattcagaatcagagcattatttttatactatgaaaatctttgaatgaagatatttaactttaaaaacatttcctaagagacaacagtgtttcttaatcattgtcttttcttcttgcagtctttcccagtgaaaacggtaaattctgctgtttgcatagaatctttaacttatttttaagatatgagattgtaaacaaattgcttgatttatttcaatcaatttattctaattatttaaataaaatcacccctaag

SEQ ID NO: 5 - protein, 1133 aa, Rattus norvegicus
MLFSLTFLSVFLKITVLSVTAQQTRNCQSGPLERSGTTTYAAAGPPRFLIFLQGNSIFRINTDGTNHQQLVVDAGVSVVMDFHYKEERLYWVDLERQLLQRVFFNGSGQETVCKVDKNVSGLAINWIDGEILRTDRWKGVITVTDMNGNNSRVLLSSLKRPANILVDPTERLIFWSSVVTGNLHRADLGGMDVKTLLEAPERISVLILDILDKRLFWAQDGREGSHGYIHSCDYNGGSIHHIRHQARHDLLTMAIFGDKILYSALKEKAIWIADKHTGKNVVRVNLDPASVPPRELRVVHLHAQPGTENRAQASDSERCKQRRGQCLYSLSERDPNSDSSACAEGYTLSRDRKYCEDVNECALQNHGCTLGCENIPGSYYCTCPTGFVLLPDGKRCHELVACPGNRSECSHDCILTSDGPLCICPAGSVLGKDGKTCTGCSFSDNGGCSQICLPLSLASWECDCFPGYDLQLDRKSCAASMGPQPFLLFANSQDIRHMHFDGTDYKTLLSRQMGMVFALDYDPVESKIYFAQTALKWIERANLDGSQRERRITEGVDTPEGLAVDWIGRRIYWTDSGKSVIEGSDLSGKHHQIIIKESISRPRGIAVHPKARRLFWTDTGMSPRIESSSLQGSDRTLIASSNLLEPSGIAIDYLTDTLYWCDTKLSVIEMADLDGSKRRRLTQNDVGHPFSLAVFEDHVWFSDWAIPSVIRVNKRTGQNRVRLRGSMLKPSSLVVVHPLAKPGADPCLHRNGGCEHICQESLGTAQCLCREGFVKAPDGKMCLTRKDDQILAGDNADLSKEVASLDNSPKAYVPDDDRTESSTLVAEIMVSGLNYEDDCGPGGCGSHAHCISEGEAAVCQCLKGFAGDGNLCSDIDECELGSSDCPPTSSRCINTEGGYVCQCSEGYEGDGIYCLDVDECQQGSHGCSENATCTNTEGGYNCTCAGCPSAPGLPCPDSTSPSLLGKDGCHWVRNSNTGCPPSYDGYCLNGGVCMYVESVDRYVCNCVIGYIGERCQHRDLRWWKLRHADYGQRHDITVVSVCVVALALLLLLGMWGTYYYRTRKQLSESSKKPSEESSSNVSSNGPDSSGAGVSSGPQPWFVVLEEHQQPKNGRLPAAGTNGAVVEAGLSSSL

SEQ ID NO: 6 - DNA, 141 bp, Rattus norvegicus
aacagtaacacaggatgcccgccgtcgtacgatgggtactgcctcaatggtggcgtgtgcatgtatgttgaatccgtggaccgctacgtgtgcaactgtgtcattggctatattggagaacgatgtcagcaccgagactta

SEQ ID NO: 7 - DNA, 1476 bp, Artificial sequence
  Synthetic polynucleotide
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccaacagtaacacaggatgcccgccgtcgtacgatgggtactgcctcaatggtggcgtgtgcatgtatgttgaatccgtggaccgctacgtgtgcaactgtgtcattggctatattggagaacgatgtcagcaccgagacttaggaattcccgaaataaaggatctttcagaaaataaacttccagttatatatatgcatgtacctaaatccggagccttaaatcaaaaagttgttttctatggaaaaggaacatatgacccagatggatctatcgcaggatatcaatgggactttggtgatggaagtgattttagcagtgaacaaaacccaagccatgtatatactaaaaaaggtgaatatactgtaacattaagagtaatggatagtagtggacaaatgagtgaaaaaactatgaagattaagattacagatccggtatatccaataggcactgaaaaagaaccaaataacagtaaagaaactgcaagtggtccaatagtaccaggtatacctgttagtggaaccatagaaaatacaagtgatcaagattatttctattttgatgttataacaccaggagaagtaaaaatagatataaataaattagggtacggaggagctacttgggtagtatatgatgaaaataataatgcagtatcttatgccactgatgatgggcaaaatttaagtggaaagtttaaggcagataaaccaggtagatattacatccatctttacatgtttaatggtagttatatgccatatagaattaatatagaaggttcagtaggaagataa

SEQ ID NO: 8 - protein, 491 aa, Artificial sequence
  Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSNSNTGCPPSYDGYCLNGGVCMYVESVDRYVCNCVIGYIGERCQHRDLGIPEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

(19) United States
(12) Patent Application Publication
(10) Pub. No.: US 2011/0281351 A1
(43) Pub. Date: Nov. 17, 2011

(54) PROCESS FOR PRODUCING LAMINATED HIGH-DENSITY CULTURED ARTIFICIAL TISSUE, AND LAMINATED HIGH-DENSITY CULTURED ARTIFICIAL TISSUE

(75) Inventors: Eijiro Adachi, Kanagawa (JP); Osamu Matsushita, Kanagawa (JP); Hironobu Iwashiro, Kanagawa (JP); Satoshi Hosoya, Kanagawa (JP); Nozomu Nishi, Kagawa (JP)

(73) Assignees: KAGAWA UNIVERSITY, Takamatsu-shi, Kagawa (JP); SCHOOL JURIDICAL PERSON THE KITASATO INSTITUTE, Minato-ku, Tokyo (JP)

(21) Appl. No.: 13/146,367

(22) PCT Filed: Jan. 28, 2010
(86) PCT No.: PCT/JP2010/051123

§ 371 Date: Jul. 26, 2011

(30) Foreign Application Priority Data
Jan. 29, 2009 (JP) 2009-017475

(51) Int. Cl. C12N 5/071

(52) U.S. Cl. 435/373; 435/325; 435/395

(57) ABSTRACT

Disclosed is a process for producing an artificial tissue, which comprises a step of providing a liquid flow control member and a mesh member in a flow path through which a cell culture liquid comprising at least one type of animal cells, a collagen-binding cell growth factor and an extracellular matrix component is circulated and cultured to accumulate the extracellular matrix molecule and the animal cells on the surface of the liquid flow control member at a high density, thereby forming a high-density cultured tissue, wherein the liquid flow control member and the mesh member are so arranged in the flow path that these members are in contact with each other or in proximity to each other, and wherein the mesh member is arranged on the back side of the liquid flow control member relative to the direction of the liquid flow. Also disclosed is an artificial tissue produced by the process.

### TECHNICAL FIELD

The present invention relates to a method of producing a high-density cultured artificial tissue, and to a high-density cultured artificial tissue. More specifically, the present invention relates to a method of producing a high-density cultured artificial tissue, including reconstituting an artificial tissue, which is more similar to a living body and is formed of two or more kinds of tissues for regenerative medicine or various experiments, such as an artificial skin or an artificial organ, within a short time, and to a laminated high-density cultured artificial tissue, which is obtained by the method.

### BACKGROUND ART

In recent years, ex vivo culture has been achieved for various cells. However, a technology for arranging those cells in three dimensions in an organic manner is applied to only a tissue having a comparatively uniform structure, such as liver. Hitherto, only the following technologies have been proposed as three-dimensional culture methods: a method including preparing an adhesion substrate (scaffold material) in advance and seeding cells thereto to culture the cells in a culture fluid (for example, JP 06-277050 A (Patent Document 1), JP 10-52261 A (Patent Document 2), JP 2001-120255 A (Patent Document 3), JP 2003-265169 A (Patent Document 4), WO 2004/078954 A1 (US 2006-147486 A1: Patent Document 5), JP 2004-65087 A (Patent Document 6), and the like); and a method including culturing a mixture of an adhesion substrate and cells on a dish (petri dish).

However, in the case of the former, the cells need to be allowed to migrate in the adhesion substrate and be kept in culture for a long period of time. In the case of the latter, the adhesion substrate is a very thin tissue, and hence the seeded cells need to be kept in culture for a long period of time until the cells cause the shrinkage of the substrate to achieve a high density. Even in the case of employing any of the above-mentioned methods, a culture period of about 2 weeks is required. During this period, the cells secrete an enzyme for decomposing the adhesion substrate, with the result that a high-density tissue once formed may be decomposed. As described above, a three-dimensionally densified cultured tissue has been expected to be useful in medical transplantation, life science experiments, clinical trials for new drugs, and the like. However, such tissue has not been widely used yet because of its prolonged production period and short available period.

Therefore, the inventors of the present invention previously proposed a method of producing a high-density cultured tissue, including: providing a mesh member and a liquid flow-controlling member in a flow path, in which a cell culture fluid containing an extracellular matrix component and animal cells is subjected to circulation culture, so that the liquid flow-controlling member is located on the back surface of the mesh member with respect to a liquid flow; and accumulating the extracellular matrix molecule and animal cells at a high density on the surface of the mesh member (WO 2006/088029 A1/EP 1857543 A1: Patent Document 7). According to this method, a high-density cultured tissue is produced and the resulting high-density cultured tissue is then collected, or subsequently, a operation of forming a different high-density cultured tissue on the above-mentioned tissue using the same or different cell culture fluid containing an extracellular matrix component and one or more kinds of animal cells is performed at least once. Thus, a laminated high-density cultured tissue in which two or more kinds of tissues are laminated can be formed. However, a specific method of forming an artificial tissue having two or more kinds of tissues laminated has not been clarified.

### CITATION LIST

### Patent Documents

[Patent Document 1] JP 06-277050 A
[Patent Document 2] JP 10-52261 A
[Patent Document 3] JP 2001-120255 A
[Patent Document 4] JP 2003-265169 A
[Patent Document 5] WO 2004/078954 A1 (US 2006-147486 A1)
[Patent Document 6] JP 2004-65087 A
[Patent Document 7] WO 2006/088029 A (EP 1857543 A1)

### SUMMARY OF INVENTION

### Technical Problem

An object of the present invention is to provide a method of producing a high-density cultured artificial tissue, including reconstituting an artificial tissue obtained by laminating two or more kinds of tissues within a short time.

### Solution to Problem

A tubular organ such as the blood vessel and digestive tract has a layered structure in which the connective tissue, smooth muscle, connective tissue, endothelial cells or epithelium cells, and the like are concentrically laminated.

The inner and outer connective tissues, which belong to the same category of the connective tissue, have:

(1) different structural components of an extracellular matrix; and
(2) even in the case of the same kind of fibroblasts, different cell growth factors to be secreted and extracellular matrix compositions depending on locations of the fibroblasts.

Those differences are caused by differences in molecular species and amount of the extra-cellular matrix or differences in type and amount of the cell growth factor.

The inventors of the present invention have confirmed that, for artificially reconstructing a tissue having those differences, there is a need of:

(1) altering cells to be embedded;
(2) altering the composition of an extracellular matrix; and
(3) altering a cell growth factor to a collagen-binding type (CBD-binding type) to prevent the cell growth factor from being diffused and distributed uniformly. Consequently, the inventors of the present invention have completed the present invention.

That is, as described below, the present invention relates to a method of producing an artificial tissue and an artificial tissue, which is obtained by the method.

1. A method of producing an artificial tissue, including culturing one or more kinds of animal cells in a cell culture fluid containing a collagen-binding cell growth factor and an extracellular matrix component.
2. The method of producing an artificial tissue according to 1 above, including producing a laminated high-density cultured artificial tissue by laminating an extracellular matrix in which the one or more kinds of animal cells are embedded, including the steps of: providing a liquid flow-controlling member (such as a poly lactic acid sheet) and a mesh member in contact with or close to each other in a flow path, in which a cell culture fluid containing one or more kinds of animal cells and an extracellular matrix component is subjected to circulation culture, so that the mesh member is located on the back surface of the liquid flow-controlling member with respect to a liquid flow; producing a high-density cultured tissue by accumulating the extracellular matrix molecule and animal cells at a high density on the surface of the liquid flow-controlling member; and subsequently performing at least once an operation of forming a different high-density cultured tissue on the tissue using a different cell culture fluid containing an extracellular matrix component and one or more kinds of animal cells, thereby forming a laminated high-density cultured tissue, in which the method includes incorporating a collagen-binding cell growth factor into a circulating culture fluid in at least one step of producing a high-density cultured tissue out of the first and subsequent steps of producing a high-density cultured tissue.
3. The method of producing an artificial tissue according to 1 or 2 above, in which the cell growth factor of the collagen-binding cell growth factor is one or two or more selected from the group consisting of an epidermal growth factor (EGF), a fibroblast growth factor (FGF), a platelet derived growth factor (PDGF), a hepatocyte growth factor (HGF), a transforming growth factor (TGF), a neurotrophic factor (NGF), a vascular endothelial growth factor (VEGF), and an insulin-like growth factor (IGF).
4. The method of producing an artificial tissue according to any one of 1 to 3 above, further including reconstructing an artificial skin using a collagen-binding epidermal growth factor (EGF-CBD) as the collagen-binding cell growth factor in combination with an epidermal cell.
5. The method of producing an artificial tissue according to 4 above, in which the reconstructing of the artificial skin includes: providing a liquid flow-controlling member and a mesh member in contact with or close to each other in a flow path, in which a cell culture fluid containing an extracellular matrix component and one or more kinds of animal cells is subjected to circulation culture, so that the mesh member is located on the back surface of the liquid flow-controlling member with respect to a liquid flow; producing a high-density dermis-like tissue through a closed circulation type high-density tissue culturing step including producing a high-density culture tissue by accumulating the extracellular matrix molecule and animal cells at a high density on the surface of the liquid flow-controlling member; and subsequently reconstructing an artificial skin using a collagen-binding epidermal growth factor (EGF-CBD) in combination with an epidermal cell.
6. The method of producing an artificial tissue according to any one of 1 to 3 above, further including reconstructing an artificial blood vessel.
7. A method of producing an artificial tissue, including the steps: providing a liquid flow-controlling member and a mesh member in contact with or close to each other, in a flow path in which a cell culture fluid containing an extracellular matrix component and one or more kinds of animal cells is subjected to circulation culture, so that the mesh member is located on the back surface of the liquid flow-controlling member in relation to a liquid flow; producing a high-density cultured tissue by accumulating the extracellular matrix molecule and animal cells at a high density on the surface of the liquid flow-controlling member; and subsequently performing at least once an operation of forming a different high-density cultured tissue on the above-mentioned tissue using a different cell culture fluid containing an extracellular matrix component and one or more kinds of animal cells, thereby forming a laminated high-density cultured artificial tissue, in which the method includes:
(1) producing a connective tissue corresponding to the capsule of the liver;
(2) laminating a neoplastic hepatic cell layer regarded as a hepatic cell on the connective tissue; and
(3) producing a layer regarded as a connective tissue in the liver to reconstruct an artificial liver.
8. The method of producing an artificial tissue according to 2, 5, or 7 above, in which the liquid flow-controlling member is a biodegradable sheet.
9. An artificial tissue, which is produced by the method according to any one of 1 to 8 above.

### Advantageous Effects of Invention

According to the present invention, the artificial tissue, which is formed of two or more kinds of tissues and is more similar to a living body, can be reconstructed within a short time.

The artificial tissue obtained in the present invention is useful in the fields of medical transplantation, new drug development, drug efficacy evaluation, infection experiments, and the like.

### BRIEF DESCRIPTION OF DRAWINGS

FIG. 1 are explanatory diagrams each showing an example of a reactor according to the present invention.

FIG. 2 is a schematic diagram of a high-strength complex artificial tissue, which can be realized by the present invention.

FIG. 3 is an explanatory diagram of a method of culturing an artificial skin under air exposure according to the present invention.

FIG. 4 is a schematic diagram of an artificial skin obtained without using a fusion protein according to the present invention.

FIG. 5 is an optical microscope image of an artificial skin prepared by the present invention.

FIG. 6 is a schematic diagram of the artificial skin prepared by the present invention.

FIG. 7 is an optical microscope image and a schematic diagram of a hepatic tissue in a living body.

FIG. 8 is a schematic diagram of an artificial hepatic tissue prepared by the present invention.

FIG. 9 is a graph showing a time-dependent change in type I collagen concentration in a circulating solution according to the present invention.

FIG. 10 is an explanatory diagram showing a method of seeding epidermal cells according to the present invention.

FIG. 11 is an electron microscope image of the artificial skin prepared by the present invention.

FIG. 12 is an optical microscope image of the artificial liver prepared by the present invention.

FIG. 13 is a graph showing a time-dependent change in albumin concentration in a culture fluid according to the present invention.

### DESCRIPTION OF EMBODIMENTS

The present invention relates to a method of producing an artificial tissue, including carrying out culture in a cell culture fluid containing a collagen-binding cell growth factor, one or more kinds of animal cells, and an extracellular matrix component. In other words, the present invention has been completed by clarifying the selection and usage of three basic factors of tissue regeneration, i.e., cells, an extracellular matrix, and a cell growth factor.

In a living body tissue, various cells express various functions in an environment filled with extracellular matrices such as collagen microfibrils at a high density. The functional expression is controlled by differences in component of an extracellular matrix and interactions mediated by various cell growth factors locally produced by various cells. However, cultured cells are present in an environment in which a network of interactions in the tissue does not function (on a plastic culture dish). Heretofore, the reconstitution of an extracellular matrix environment has been achieved (Patent Document 7: WO 2006/088029 A1). However, the reproduction of cell-cell interactions with a group of cell growth factors in the tissue has not been attained.

The living body tissues have different networks of cell-cell interactions with a group of cell growth factors even in the case of the tissues of the same kind. Many of the cell growth factors are soluble proteins, and hence disperse and lose their physiological actions even if the cell growth factors are directly administered to an artificial tissue. In the tissue, the cell growth factors are produced by cells as needed and are secreted to an extracellular space or exist in the forms of being bound to extracellular structures. As examples of the latter, in the living body tissue, Latent TGF-β binds to extracellular fibrillin microfibrils and fibroblast growth factor (FGF) binds to a basal membrane as an extracellular structure, which are present as cell growth factors in an inactive state. In reconstituting the living body structure as described above, the method of the present invention includes reconstituting not only an extracellular matrix environment but also cell-cell interactions with a group of cell growth factors in a simultaneous manner by use of fusion proteins of collagen-binding domains (CBDs) and various cell growth factors.

For example, when an artificial blood vessel made from Dacron fiber is transplanted into the aorta, fibroblasts, smooth muscle cells, vascular endothelial cells, and the like move onto the transplanted material and proliferate to reconstitute (reconstruct) a blood vessel wall formed of three layers of tunica externa, tunica media, and tunica interna. However, in such artificial blood vessel, it requires a long period of time for cells to cover the surface of the transplanted blood vessel and reconstruct the tissue. Therefore, there are problems in that a thrombus is formed on the surface of an incomplete blood vessel, a long artificial blood vessel is hardly transplanted, and the like. The artificial tissue of the present invention may be prepared from cells from a patient him/herself to prevent onset of an immune rejection response, and hence may be suitably used as a material for transplantation. According to the method of the present invention, an extensive increase in survival rate of a transplanted tissue can be expected by reconstructing the basic structure of a patient's tissue in advance.

According to the present invention, a cancer tissue may also be reconstituted. Therefore, the susceptibility of a cancer tissue reconstituted from patient's own cancer cells to an anti-cancer drug can be more correctly searched.

New drug development and infection experiments are conducted using cells seeded on a plastic culture dish. However, cultured cells and in vivo cells differ in functional expression from each other even if they are of the same cell type. The present invention allows a three-dimensional cultured tissue to be more simply supplied in a short time, it can be expected that such tissue can be used in the new drug development and infection experiments.

Many of the infection experiments are conducted using laboratory animals such as rats and mice. In those animals, their inherent immune systems work and eliminate infected microorganisms such as bacteria and viruses. Leucocytes, dendritic cells that present antigens which are invading foreign substances, and the like are responsible for the biological responses. Those cells are not found in the artificial tissue while, in many cases, an immune system eliminates infected cells from the tissue. In the artificial tissue, therefore, it is expected that responses of cells infected by microorganisms can be analyzed in more detail. Further, immune responses may be analyzed by incorporating part or all of dendritic cells or leucocytes into the tissue at the time of tissue reconstitution.

[Collagen-Binding Cell Growth Factor]

According to the previous application (Patent Document 7: WO 2006/088029 A1), a uniform artificial tissue, which corresponds to the dermis of the skin or the capsule of liver, can be obtained by dispersing and circulating cells in a molecular collagen solution and laminating the cells while polymerization of collagen is controlled. The present invention includes providing cell growth factors as a solid phase in a specific layer (for example, an upper layer, a middle layer, or a lower layer) in the artificial tissue to impart a specific function, and inducing the differentiation and proliferation of cells in close contact with collagen in the layer in a specific direction. It is also possible to impart a specific function such as an anti-inflammatory function to a specific layer. This is realized by using a fusion protein of a cell growth factor and a protein capable of binding to insoluble collagen, or part thereof, i.e., a collagen-binding domain, to provide the cell growth factor as a solid phase on the polymerized insoluble collagen. In other words, the functions of the tissue can be reproduced by fusing the cell growth factor with part of a protein that specifically binds to collagen or collagen microfibrils, i.e., a collagen-binding domain (CBD).

Hereinafter, there is described a method of preparing a collagen-binding type epidermal growth factor (EGF-CBD) as an example of collagen-binding cell growth factors which may be used for the above-mentioned purposes.

[Method of Preparing Collagen-Binding Type Epidermal Growth Factor (EGF-CBD)]

The fusion protein is prepared through the following three steps:

(1) constructing an expression vector having inserted therein a gene fragment that encodes a collagen-binding domain (CBD) of bacterial collagenase;

(2) constructing an expression plasmid that encodes EGF-CBD by insertion of a gene fragment that encodes epidermal growth factor (EGF) into the expression vector according to the item (1); and

(3) transforming the expression plasmid according to the item (2) into host cells, and producing and purifying a fusion protein.

Hereinafter, those steps are described in detail.

(1) Step of Constructing Expression Vector Having Inserted Therein Gene Fragment that Encodes Collagen-Binding Domain (CBD) of Bacterial Collagenase

A DNA fragment that encodes a collagen-binding domain is obtained by a PCR method or the like using a structural gene of known bacterial collagenase as a template. Then, the desired expression vector may be obtained by a method of inserting the DNA fragment into any expression vector (e.g., a pGEX-4T vector that produces a protein of interest as a fusion protein with glutathione S transferase (GST)) by an ordinary method.

An exemplary collagenase structural gene is DNA (SEQ ID NO: 1) of Clostridium histolyticum colH (GenBank Accession No. D29981). The amino acid sequence of collagenase encoded by the DNA is set forth in SEQ ID NO: 2. Of those, DNA that encodes the collagen-binding domain corresponds to DNA (SEQ ID NO: 3) formed of a base sequence of base Nos. 3010 to 3366 in SEQ ID NO: 1. However, the sequence may have variations and deletions in the normally acceptable range. Alternatively, as long as such region is included, another region may be included in the normally acceptable range.

(2) Step of Constructing Expression Plasmid that Encodes EGF-CBD by Insertion of Gene Fragment that Encodes Epidermal Growth Factor (EGF) into Expression Vector According to Item (1)

A cDNA library, which is prepared from total RNA obtained from EGF-expressing cells by an ordinary method, is used as a template to obtain a DNA fragment that encodes an epidermal growth factor by a PCR method or the like. After that, the DNA fragment may be inserted into the expression vector according to the item (1) by an ordinary method, thereby obtaining the desired expression plasmid. The cells are preferably ones derived from mammals, and in particular, are most preferably ones derived from humans.

An exemplary structural gene of epidermal growth factor is cDNA (SEQ ID NO: 4) of Rattus norvegicus preproEGF (GenBank Accession No. U04842). The amino acid sequence of preproEGF encoded by the DNA is set forth in SEQ ID NO: 5.

(3) Step of Introducing Expression Plasmid According to Item (2) into Host Cells, and Producing and Purifying Fusion Protein

Any kind of host cells may be used as long as the host cells corresponds to the expression vector used. For example, if the expression vector is a prokaryotic vector, prokaryotic cells may be used. If the expression vector is an insect vector, insect cells may be used. Further, the introduction of the expression vector may be performed by an ordinary method such as an electroporation method or a calcium method.

Cell culture and fusion protein production are carried out by methods suitable for transformed cells and an expression vector. For example, when a vector expressing a fusion protein of EGF-CBD with glutathione S transferase (GST) or His tag is used as the expression vector, the isolation and purification of EGF-CBD from a culture can be easily performed using a known affinity purification method suitable for such fusion protein. It should be noted that cutting only EGF-CBD out of such fusion protein and further removing the tag therefrom may be also performed using a known method.

It should be noted that EGF-CBD as a substance has been known in the Document (Nishi N, Matsushita 0, et al., Proc Natl Acad Sci U S A. 95:7018-7023. 1998), but the Document describes that EGF-CBD did not show an expected effect in an animal experiment.

In the same manner as described above, another collagen-binding cell growth factor can be prepared as a fusion protein.

Examples of the collagen-binding cell growth factor include, but not particularly limited to, a collagen-binding epidermal growth factor (EGF-CBD), a collagen-binding fibroblast growth factor (FGF-CBD), a collagen-binding platelet derived growth factor (PDGF-CBD), a collagen-binding hepatocyte growth factor (HGF-CBD), a collagen-binding transforming growth factor (TGF-CBD), a collagen-binding neurotrophic factor (NGF-CBD), a collagen-binding vascular endothelial growth factor (VEGF-CBD), and a collagen-binding insulin-like growth factor (IGF-CBD).

[Closed Circulation Type High-Density Tissue Production Apparatus (Reactor)]

According to the present invention, in a method of producing laminated high-density cultured artificial tissue by laminating extracellular matrices having embedded therein one or more kinds of animal cells includes the steps of: providing a liquid flow-controlling member and a mesh member in a flow path, in which a cell culture fluid containing one or more kinds of animal cells and an extracellular matrix component is subjected to circulation culture, so that the mesh member is located in contact with or close to the liquid flow-controlling member on the back surface thereof with respect to a liquid flow; producing a high-density cultured tissue by accumulating extracellular matrix molecules and animal cells at a high density on the surface of the liquid flow-controlling member; and subsequently performing at least once an operation of forming a different high-density cultured tissue on the tissue using a different cell culture fluid containing an extracellular matrix component and one or more kinds of animal cells, a collagen-binding cell growth factor can be incorporated in a circulation culture fluid in at least one step of producing a high-density culture tissue out of the first and subsequent steps of producing a high-density culture tissue, thereby producing an artificial tissue.

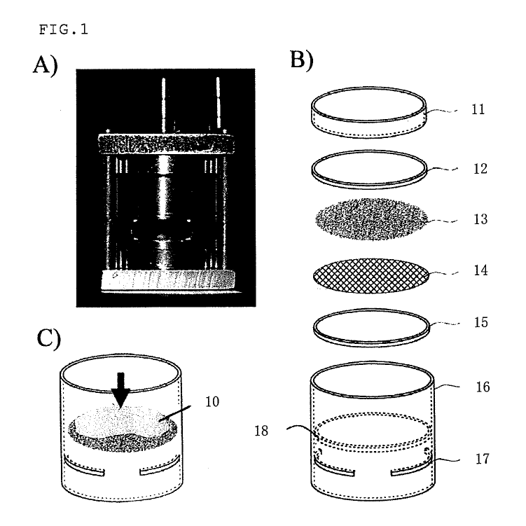

The artificial tissue can be reconstituted by changing a combination of the one or more kinds of animal cell species and the extracellular matrix component. For example, a biodegradable sheet made of polylactic acid or the like is attached in the inside of a laminated high-density cultured tissue production apparatus (which may be referred to as “closed circulation type high-density tissue culture apparatus” or simply referred to as “reactor”) for circulation culture of the cell culture fluid (FIG. 1). A culture suspension containing a collagen protein and fibroblasts is circulated through the sheet in the reactor, and the collagen microfibrils formed during the circulation and fibroblasts are deposited on the biodegradable sheet attached in the reactor, thereby forming an artificial connective tissue. Next, a culture suspension containing second cells and a second extracellular matrix component can be circulated to laminate a second tissue on the connective tissue, thereby reconstructing a tissue. Similarly, a desired number of tissues can be laminated and reconstructed as an artificial tissue.

In the present invention, the use of a biodegradable sheet made of a polylactic acid sheet (PLA sheet) or the like as a liquid flow-controlling member allows collagen microfibrils to be reconstructed on the surface of the sheet due to its permeability and local circulation control. Therefore, it is possible to simplify the structure of the reactor and simultaneously prevent the circulation failure of the reactor due to clogging when filter paper is used as a local circulation-controlling material. As a result, a complex tissue including several layers can be prepared by various combinations of three factors, i.e., the extracellular matrix composition of a circulating culture fluid, the kind of cells to be suspended, and a fusion protein formed of a cell growth factor and a collagen-binding domain, depending on tissues of interest. Further, an entry flow path for nutrition blood vessels in the artificial tissue can be provided by sandwiching the connective tissue between layers of functional cells such as epithelial cells and smooth muscle cells.

[Artificial Tissue]

In general, a tissue has a structure with the following features:

(1) tissues having different functions are arranged in layers; and
(2) a plurality of cells are located in a high-density extracellular substance (extracellular matrix) such as collagen microfibrils in each of the tissues.

The basic structure can be reproduced by laminating high-density extracellular substances having embedded therein various cells. A technology that makes it possible is the method of the present invention using a “closed circulation type high-density tissue culture apparatus” (reactor). In order that a tissue to be reconstructed exhibits a specific function of interest, there is a need of cells having such function and a cell growth factor for facilitating the expression of such function. Many cell growth factors are produced in the tissue and express their functions in the tissue. Thus, for example, a method of incorporating a gene that encodes a specific functional protein into cells by a genetic engineering technique has been attempted. However, it is difficult to control the amount of a protein produced by the introduced gene and the application thereof is restricted in view of a risk of tumorigenesis and the like.

[Production of Artificial Tissue]

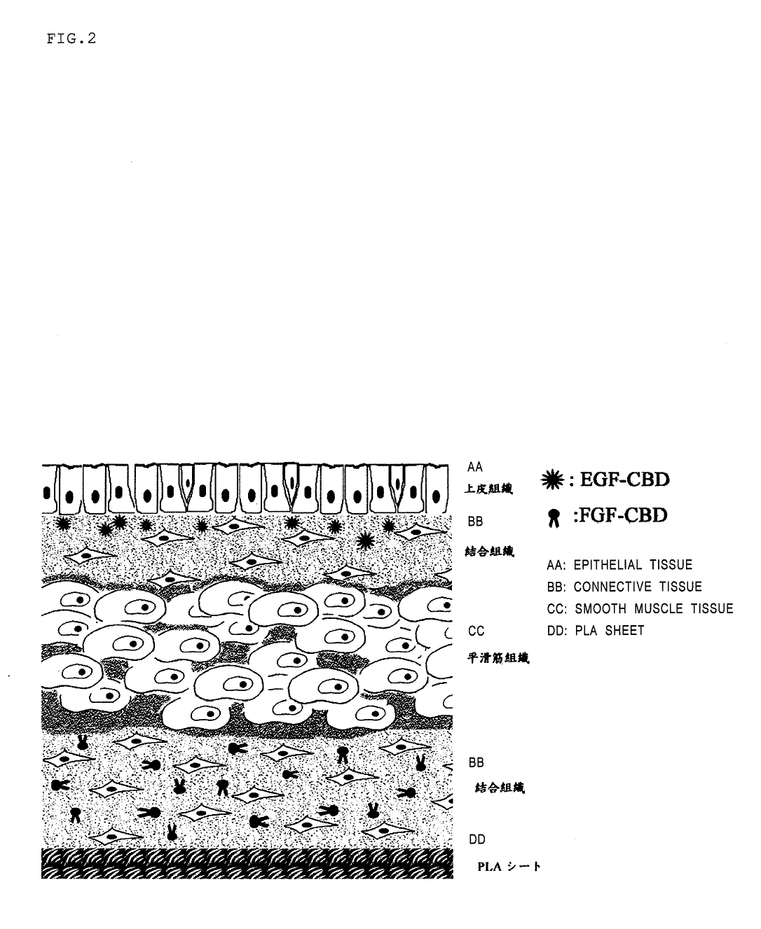

A method of producing an artificial tissue is descried using the digestive tract and blood vessel as models with reference to FIG. 2.

(1) A culture fluid containing collagen, one or more kinds of animal cells, and a collagen-binding cell growth factor is subjected to circulation culture to reconstitute a first tissue (connective tissue).

In other words, an appropriate amount of DMEM (culture fluid) containing an appropriate concentration of collagen of each type, human fibroblasts or pluripotent stem cells, and a fusion protein as a combination of an appropriate concentration of a fibroblast growth factor (FGF) and a collagen-binding domain (CBD) is circulated in a closed circulation type high-density tissue production apparatus for 4 to 6 hours. This also provides a passage for the blood vessel or nerve. Thus, proteins of the vascular endothelial growth factor (VEGF) and nerve growth factor (NGF) coupled with CBD are incorporated, thereby forming a connective tissue such as an outer membrane in, for example, the digestive tract.

(2) A different culture fluid containing one or more kinds of animal cells and a membrane component is subjected to circulation culture to reconstitute a second tissue (smooth muscle tissue).

In other words, an appropriate amount of DMEM is circulated for about 1 hour. Subsequently, a necessary amount of DMEM containing smooth muscle cells or pluripotent stem cells and a basal membrane component adjusted to an appropriate concentration is added to the circulating solution and the mixture is circulated for about 2 hours. This operation forms a tissue called “tunica media” in the digestive tract or the blood vessel.

(3) A different culture fluid containing collagen, one or more kinds of animal cells, and a collagen-binding cell growth factor is subjected to circulation culture to reconstitute a third tissue (connective tissue).

In other words, an appropriate amount of DMEM (culture fluid) containing appropriate concentrations of type III and type V collagens, human fibroblasts or pluripotent stem cells, and an appropriate concentration of FGF-CBD is circulated in a closed circulation type high-density tissue production apparatus for about 2 hours. This operation forms a tissue called “tunica interna” in the digestive tract or the blood vessel.

(4) A different culture fluid containing one or more kinds of animal cells is subjected to circulation culture to reconstitute a fourth tissue (epithelial tissue).

In other words, the culture fluid is replaced with a culture fluid in which endothelial cells for the blood vessel and epithelial cells for the digestive tract are suspended alone or in combination with pluripotent stem cells and then circulated for about 2 hours. In addition, in the case of a comparatively uniform tissue such as the cartilage tissue, an appropriate amount of DMEM (culture fluid) containing appropriate concentrations of type II collagen and human cartilage cells or pluripotent stem cells is circulated in a closed circulation type high-density tissue production apparatus for 4 to 6 hours, thereby forming the cartilage tissue.

[Artificial Skin]

In the conventional production of an artificial skin, first, a mixed liquid of fibroblasts and collagen is kept at a neutral pH at 37° C. to produce a low-density dermis-like tissue. When epidermal cells are seeded on the low-density collagen gel, the cells sink into the gel. Thus, there is a need of forming a high-density dermis-like tissue, in which the gel shrinks to a size of 1/10 of the original size of the gel by the action of fibroblasts confined by culturing the gel in the culture fluid for 3 to 7 days (hereinafter, referred to as “contracted gel”). In the present invention, however, a high-density dermis-like tissue can be obtained by the reactor in about 6 hours, and immediately, epidermal cells can be seeded. In the contracted gel, part of the basal membrane components and a cell growth factor are secreted from the fibroblasts in the culture of 3 to 7 days, and an environment suitable for proliferation of epidermal cells is established. However, fibroblasts in the contracted gel also secrete a matrix metalloprotease, which decomposes collagen microfibrils, simultaneously, and hence the autolysis of the resulting artificial skin is also quick. Therefore, there is a disadvantage in that the life period of the artificial skin is short.

In order to solve this problem, the present invention provides a method including:

(1) producing a high-density dermis-like tissue within a short time by using a reactor; and then (2) reconstituting an epidermal layer using a fusion protein as a combination of a cell growth factor and a collagen-binding domain (CBD), in combination with epidermal cells.

In other words, the present invention includes:

(1) providing a liquid flow-controlling member and a mesh member in contact with or close to each other, in a flow path in which a cell culture fluid containing one or more kinds of animal cells and an extracellular matrix component is subjected to circulation culture, so that the mesh member is located on the back surface of the liquid flow-controlling member with respect to a liquid flow, and producing a high-density dermis-like tissue by a closed circulation type high-density tissue culturing step of producing a high-density cultured tissue by accumulating extracellular matrix molecules and animal cells at a high density on the surface of the liquid flow-controlling member; and then
(2) reconstructing an artificial skin using a collagen-binding cell growth factor in combination with epidermal cells.

[Production of Artificial Skin]

An artificial skin can be produced, for example, according to the following items (1) to (4).

(1) 200 mL of DMEM (culture fluid) containing 0.5 mg/mL atelocollagen (I-AC; KOKEN Co., Ltd.) and human fibroblasts (HFO; 2×10⁷ cells) are circulated in a closed circulation type high-density tissue production apparatus for 6 hours.
(2) An artificial dermis tissue was taken out from the apparatus, cultured for 1 week in 2 mL of DMEM supplemented with ascorbic acid 2-glucopyranose (AA2G: 84.3 mg/mL), and further cultured for 1 week in DMEM supplemented with the same concentration of ascorbic acid 2-glucopyranose and a synthetic matrix metalloprotease inhibitor (CGS: 10 mM)
(3) A cylinder made of glass with 10.5 mm in inner diameter and 5 mm in height is placed on the artificial dermis tissue. Then, a mixed culture fluid (0.4 mL) containing DMEM in which EGF-CBD (0.95 μg/mL) and cultured epidermal cells (4×10⁵ cells) are suspended and a human epidermal growth factor (hEGF)-free Epi-life (1:1) is poured into the cylinder. It is confirmed that there is no leakage from the cylinder and the mixture is then cultured overnight.
(4) The cylinder is removed and the entire artificial skin is pulled up. Then, culture is performed while the upper portion of the artificial skin is exposed to the air and the culture fluid used in the item (2) is replaced every 2 days (FIG. 3).

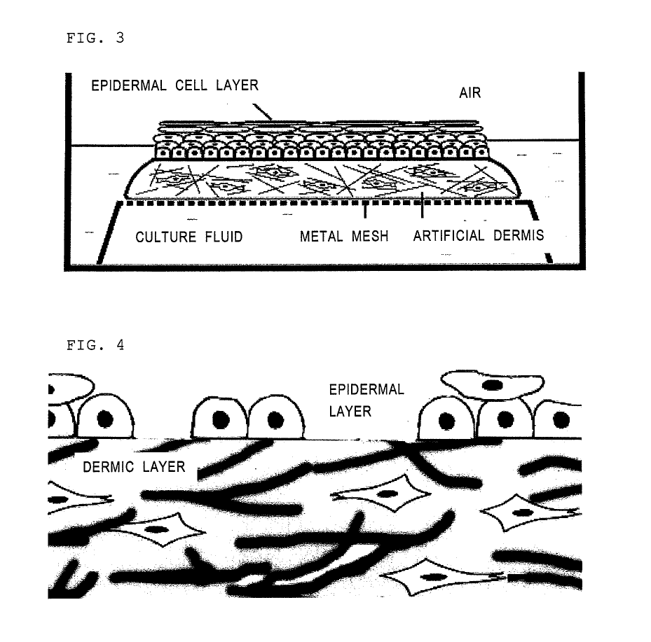

When the above-mentioned fusion protein is not used, proliferation of epidermal cells is insufficient and a multi-layered epidermal tissue cannot be obtained (FIG. 4). According to the present invention, epidermal layers of five or six layers can be reconstituted by seeding the above-mentioned fusion protein in combination with epidermal cells.

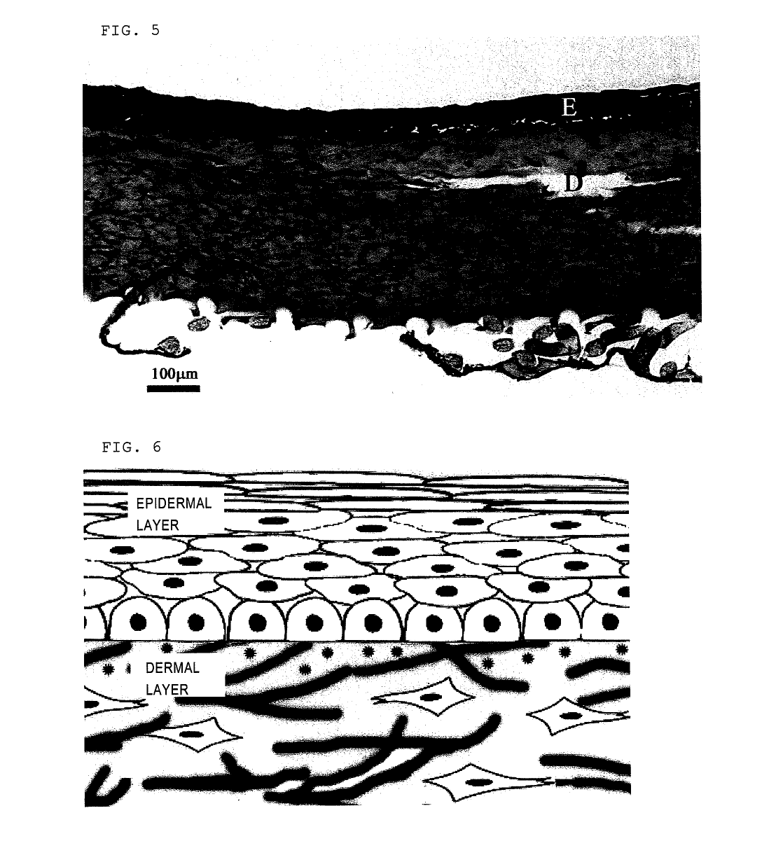

Typically, however, a skin tissue, as shown in an optical microscope image thereof of FIG. 5, can be reconstituted by addition of a fusion protein (hereinafter, referred to as “EGF-CBD”) (0.95 μg/mL), which is a combination of an epidermal growth factor (EGF) and a collagen-binding domain (CBD) of bacterial collagenolytic enzyme, to an epidermal cell suspension. FIG. 6 is a schematic diagram of the skin tissue. EGF-CBD presumably promotes the proliferation of the seeded cultured epidermal cells for a long period of time by binding to collagen microfibrils present in the upper part of a high-density dermis-like tissue produced using the reactor. It is considered that the epithelial cell growth factor free of a collagen-binding domain may diffuse in a culture fluid and the concentration thereof may be reduced to a concentration equal to or lower than one for facilitating the proliferation of epidermal cells. As the epidermal cells, already matured somatic epidermal cells may be seeded. Alternatively, a mixture of the epidermal cells with stem cells and pluripotent stem cells, which may be easily proliferated, such as iPS cells may be seeded. In general, somatic epidermal cells have a low proliferation rate, and hence it takes much time for obtaining a sufficient number of epidermal cells. The action of EGF-CBD may facilitate differentiation of stem cells mixed with the somatic cells.

[Artificial Liver]

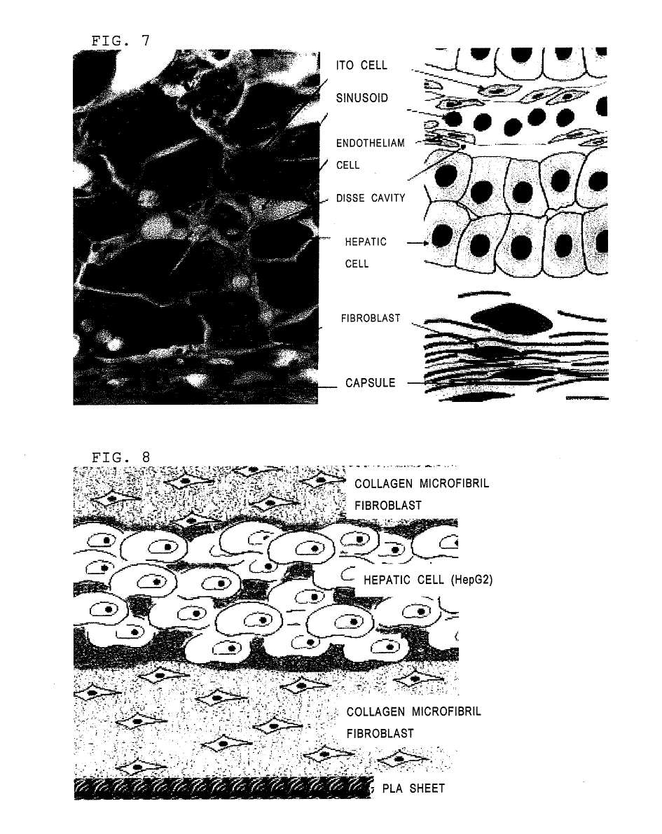

The liver is covered with a connective tissue called a capsule (see an optical microscope image of FIG. 7). First, the connective tissue corresponding to the capsule is produced in the reactor. Next, neoplastic hepatic cell (HepG2) layers regarded as hepatic cells are laminated. Finally, a layer regarded as the connective tissue in the liver is produced (FIG. 8).

In other words, according to the present invention, an artificial liver may be produced by a method of forming a laminated high-density cultured artificial tissue, including the steps of: providing a liquid flow-controlling member and a mesh member in contact with or close to each other in a flow path, in which a cell culture fluid containing an extracellular matrix component and one or more kinds of animal cells is subjected to circulation culture, so that the mesh member is located on the back surface of the liquid flow-controlling member with respect to a liquid flow, and producing a high-density cultured tissue by accumulating the extracellular matrix molecule and animal cells at a high density on the surface of the liquid flow-controlling member; and subsequently performing at least once an operation of forming a different high-density cultured tissue on the tissue using a different cell culture fluid containing an extracellular matrix component and one or more kinds of animal cells, in which the method includes: (1) producing a connective tissue corresponding to the capsule of the liver; (2) laminating thereto a neoplastic hepatic cell layer regarded as a hepatic cell; and (3) producing a layer regarded as a connective tissue in the liver.

For liver regeneration, it has hitherto been focused on how to arrange hepatic cells in a three-dimensional manner. However, unlike the present invention, there is no approach focused on the fact that the morphology of the liver is maintained by a connective tissue structure, such as a capsule or Glisson's capsule. The human liver weighs about 1.4 kg and is formed of 1.5×10¹² cells. To make these many cells functionally configured in three dimensions, the cells need to be supported by the connective tissue. The artificial liver according to the present invention is produced by imitating the liver structure in the living body. Thus, the method makes it possible to produce a large-sized artificial liver.

[Production of Artificial Liver]

An artificial liver can be produced, for example, according to the following items (1) to (5).

(1) 100 mL of DMEM (culture fluid) containing 0.5 mg/mL type I atelocollagen (I-AC; KOKEN Co., Ltd.) and human fibroblasts (HFO; 1 to 2×10⁷ cells) are circulated in a closed circulation type high-density tissue production apparatus for 6 hours.
(2) The medium is replaced with 50 mL of DMEM, and immediately after starting the circulation, 2 mL of a suspension of HepG2 cells (2 to 4×10⁷ cells) are completely loaded from the upstream of the reactor over 5 to 10 minutes.
(3) DMEM (50 mL) is circulated for 2 hours.
(4) 50 mL of DMEM (culture fluid) containing 0.5 mg/mL atelocollagen (I-AC; KOKEN Co., Ltd.) are circulated for 3 hours.
(5) The completed laminated artificial hepatic tissue is transferred to a circulation culture apparatus and subjected to circulation culture in DMEM containing 10% fetal bovine serum for 3 days.

In the present invention, in a flow path, in which a cell culture fluid containing an extracellular matrix component and one or more kinds of animal cells is subjected to circulation culture, a liquid flow-controlling member and a mesh member are provided so that the members are in contact with or close to each other. In this case, it is preferred that the liquid flow-controlling member be arranged on the upstream with respect to the flow of the culture fluid to accumulate the extracellular matrix molecule and animal cells at a high density on the surface of the liquid flow-controlling member.

In the above-mentioned flow path, in which the above-mentioned culture fluid is subjected to circulation culture, the liquid flow-controlling member and the mesh member are provided so that the members are in contact with or close to each other. Thus, the flow rate of the culture fluid can be lowered locally, and the concentrations of the extracellular matrix component and the animal cells suspended in the cell culture fluid can be increased locally. As a result, the extracellular matrix molecule and the animal cells can be accumulated at a high density on the liquid flow-controlling member.

In order to perform uniform high-density accumulation of the extracellular matrix molecule and animal cells, the culture fluid should flow almost constantly on the liquid flow-controlling member and the mesh member. In one embodiment, the uniform high-density accumulation is realized by using planar members as the liquid flow-controlling member and the mesh member, arranging the members in parallel with each other, and allowing the culture fluid to flow in the direction almost perpendicular to the surface of the liquid flow-controlling member. In another embodiment, the uniform high-density accumulation is also realized by using cylindrical members as the liquid flow-controlling member and the mesh member, concentrically arranging the members so that the liquid flow-controlling member is located inside, and allowing the culture fluid to flow from the inner side towards the outer side of the liquid flow-controlling member. Alternatively, other embodiments are also possible (Patent Document 7: WO 2006/088029 A1).

In particular, preferred is an embodiment in which the culture fluid is allowed to flow from the side of the liquid flow-controlling member with respect to the planer liquid flow-controlling member and mesh member provided in parallel with each other. Such embodiment is realized, for example, by installing, in a flow path, a stainless-steel cylinder (16) having a plurality of slits (17) in the lower portion thereof as shown in FIG. 1.

In this example, a PLA sheet (13) is provided in the stainless-steel cylinder (16). A stainless-steel mesh (14) is provided below the sheet. Preferably, the stainless-steel cylinder (16) has a flange (18) on the inner periphery thereof, and if required, one leakage-preventing member (e.g., a silicon rubber ring) (12) is placed on the PLA sheet (13) and another leakage-preventing member (15) is placed below the stainless-steel mesh (14). Further, for example, a spacer (11) is placed as a liquid leakage-preventing member. FIG. 1 each show a state in which those members are removed. During use, however, those members are attached so that they are fixed with the flange (18) in the stainless-steel cylinder (16) and installed in the flow path.

The overall configuration of the apparatus is, for example, a closed circulation type culture apparatus in which a reactor body, a medium reservoir, a circulating pump, and a flow cell are connected to one another through pipe lines and installed in an incubator. Preferably, the apparatus includes a sensor such as a dissolved oxygen (DO) sensor, a display device for displaying a measured value thereof, and a stirrer for stirring a medium in the medium reservoir. The stirrer is, for example, a magnetic rotation device for spinning a magnetic stirring bar placed in the medium reservoir.

It should be noted that, as the overall configuration of the exemplary apparatus described above, one described in Patent Document 7 (WO 2006/088029 A1) may be employed.

The liquid flow-controlling member is not particularly limited as long as it is a member capable of allowing a liquid flow to pass through and reducing the rate of the flow. In general, however, it is a liquid flow permeable porous material, particularly a liquid flow permeable porous membrane. Examples of such membrane include filter paper, a woven fabric, a nonwoven fabric, a silk fibroin membrane, and a biodegradable sheet. Of those, a biodegradable sheet such as a polylactic acid sheet (PLA sheet) is preferred.

The mesh member is generally a member having a mesh size which does not extensively prevent a liquid flow. Specifically, the mesh member has a pore size of about 100 μm to 1 mm, more preferably about 100 μm to 0.5 mm. For example, a mesh having a pore size of about 100 μm to 300 μm, which is formed by weaving wires of about 0.08 to 0.1 mm in diameter, may be used. Materials for the mesh member may be any of metals (e.g., stainless-steel), synthetic resins (e.g., polyester), ceramics, artificial materials, and the like. Usually, a metal mesh is preferred in the light of sterilization and facilitated washing operation.

In the apparatus (reactor) of the present invention, the liquid flow-controlling member and the mesh member are provided in contact with or close to each other. Here, the term “close” means that the stagnation of a solution by the liquid flow-controlling member may be caused in the vicinity of the mesh member and generally means a distance of about several millimeters (mm) or less, preferably about 1 mm or less. Any of the liquid flow-controlling member and the mesh member may be arranged on the upstream side (viewing from the liquid flow). In the case where the liquid flow-controlling member is arranged on the upstream side, a complex member including the high-density cell cultured tissue formed of an extracellular matrix component and animal cells and the liquid flow-controlling member can be obtained. Further, the liquid flow-controlling member and the mesh member may be unified.

Dimensional conditions other than those described above of the liquid flow-controlling member and the mesh member (an area or a diameter in the case of a radial flow type reactor) may largely depend on the kind of cells and the size of a tissue, which are to be grown up. In the vicinity of the liquid flow-controlling member or the mesh member, the circulation rate of the cell culture fluid may be, for example, about 4 to 10 μL/cm²/sec, preferably about 6 to 8 μL/cm²/sec.

In the apparatus of the present invention, the extracellular matrix component contained in the cell culture fluid may be any molecule as long as it is polymerizable or mutually bindable as a cell adhesion substrate at 37° C. and in a neutral pH region. Typically, the extracellular matrix component is a substance found in the connective tissue. Examples of such substance include collagen, elastin, proteoglycan, fibrillin, fibronectin, laminin, chitin, and chitosan. Those extracellular matrix components may be used alone or may be used as a combination of two or more kinds thereof. Further, each of the above-mentioned components may be subjected to various kinds of chemical modification. The modification may be one typically found in the living body or may be artificial modification for imparting various activities and characteristics. Further, constituents of each of the above-mentioned components may be also included (e.g., for proteoglycan, glycosaminoglycans such as hyaluronic acid, chondroitin sulfate, dermatan sulfate, heparan sulfate, heparin, and keratan sulfate).

The extracellular matrix component is preferably collagen or elastin or a combination of collagen or elastin with one or more kinds of the above-mentioned components, particularly preferably a combination of collagen or collagen with one or more kinds of the above-mentioned components. Preferred components are determined depending on the type of a cultured tissue of interest.

The collagen may be any kind of conventionally known collagens. For example, type I, type II, type III, type IV, and type V collagens and the like may be used.

Such collagen may be one obtained by using, as a raw material, a living body tissue containing collagen to be obtained and solubilizing the living body tissue with an acid, an enzyme, an alkali, or the like. In addition, to avoid or inhibit an allergic response and a rejection response, it is preferred to completely or partially remove a telopeptide on the terminal of a molecule by an enzyme treatment. Examples of such collagen material include type I collagen from porcine skin, type I collagen from porcine tendon, type II collagen from bovine nasal cartilage, type I collagen from fish, genetically engineered collagen, and mixtures thereof. However, those are provided for the purposes of illustration and other kinds of collagens may be available depending on purposes. For example, type IV is used in the case of forming the tissue corresponding to the basal membrane.

The animal cells contained in the cell culture fluid are appropriately selected depending on purposes and are not particularly limited. Examples of the animal cells include somatic cells, tumor cells, and embryonic stem cells. Examples of the somatic cells include fibroblasts, hepatic cells, vascular endothelial cells, epidermal cells, epithelial cells, cartilage cells, neuroglia cells, and smooth muscle cells. Those may be used alone or as a mixture of two or more kinds thereof.

Although the basic composition of the cell culture fluid vary depending on the kind of animal cells to be cultured, a conventional natural medium or synthetic medium may be used. In consideration of infection of bacteria, viruses, or the like from animal-derived substances, variations in compositions due to supply dates and places, and the like, a synthetic medium is more preferred. Examples of the synthetic medium include, but not particularly limited to, an α-minimum essential medium (α-MEM), Eagle MEM, Dulbecco MEM (DMEM), an RPMI1640 medium, a CMRC medium, an HAM medium, a DME/F12 medium, a 199 medium, and an MCDB medium. Commonly used serum and the like may be added as appropriate. Examples of the natural medium include, but not particularly limited to, conventionally known natural media. Those may be used alone or may be used in combination of two or more kinds thereof.

The content of the extracellular matrix component in the cell culture fluid is about 0.1 to 0.5 mg/mL, preferably about 0.2 to 0.3 mg/mL at the time of onset of culture.

It should be noted that the cell culture fluid may contain, in addition to the above-mentioned extracellular matrix component, other substances that facilitate cell adhesion, including: peptides and proteins such as polylysine, histone, gluten, gelatin, fibrin, and fibroin; cell-adhesive oligopeptides such as RGD, RGDS, GRGDS, YIGSR, and IKVAV, or synthetic proteins having incorporated thereinto the sequences thereof through a genetic engineering technique; polysaccharides such as alginic acid, starch, and dextran, and derivatives thereof; biodegradable polymers such as polymers and copolymers of lactic acid, glycolic acid, caprolactone, and hydroxybutyrate, and a block copolymer of any such polymer or copolymer with polyethylene glycol or polypropylene glycol.

Further, the culture fluid may also contain a biologically active substance other than those described above. Examples of the biologically active substance include cell growth factors, hormones, and/or natural or synthetic chemical substances having pharmacological actions. The addition of such substance may impart an additional function to the culture fluid or may change functions of the culture fluid. Further, a cell-incorporated tissue containing a synthetic compound, which does not exist in nature, can be obtained by modifying circulation conditions.

Examples of the cell growth factor include, but not particularly limited to, an epidermal growth factor (EGF), a fibroblast growth factor (FGF), a platelet-derived growth factor (PDGF), a hepatocyte growth factor (HGF), a transforming growth factor (TGF), a neurotrophic factor (NGF), a vascular endothelial growth factor (VEGF), and an insulin-like growth factor (IGF). Other cell growth factors may also be used depending on the kind of cells to be cultured.

Examples of the hormone include, but not particularly limited to, insulin, transferrin, dexamethasone, hydrocortisone, thyroxine, 3,3′,5-triiodothyronine, 1-methyl-3-butylxanthine, and progesterone. Those hormones may be used alone or may be used in combination of two or more kinds thereof.

Examples of the other biologically active substances include vitamins such as ascorbic acid (in particular, L-ascorbic acid), biotin, calcium pantothenate, and ascorbic acid 2-phosphate, and vitamin D, proteins such as serum albumin and trans ferrin, lipids, fatty acid, linoleic acid, cholesterol, pyruvic acid, nucleotides for synthesizing DNA and RNA, glucocorticoid, retinoic acid, β-glycerophosphate, monothioglycerol, and various antibiotics. It should be noted that those substances are given merely as examples, and other components may be used depending on the purposes. The above-mentioned components may be used alone or may be used in combination of two or more kinds thereof.

Culture may be performed under normal conditions until a high-density cultured tissue with a desired size (thickness) is generated. Typically, the culture temperature is 35 to 40° C. and the culture time is 6 hours to 9 days. As described above, the conventional method of producing a high-density cultured tissue requires 2 or more weeks. According to the apparatus of the present invention, a required culture time is shortened greatly.

Further, according to the apparatus of the present invention, there is provided a method of producing a high-density cultured tissue, including: producing a high-density cultured tissue by any one of the methods described above; collecting the resulting high-density cultured tissue; and culturing the tissue continuously in a non-circulating culture fluid with the same or different formulation containing an extracellular matrix component and one or more kinds of animal cells. Here, the non-circulating culture condition refers to, for example, culture on a dish. By employing such method, it is expected that newly laminated cells can proliferate and differentiate in a state similar to the living body.

Further, according to the apparatus of the present invention, after producing a high-density cultured tissue by any one of the methods described above, the resulting high-density cultured tissue is collected, or subsequently, an operation of forming a different high-density cultured tissue on the tissue using the same or different culture fluid containing an extracellular matrix component and one or more kinds of animal cells can be performed at least once to form a laminated high-density cultured tissue.

Further, according to the apparatus of the present invention, for example, it is possible to carry out culture while continuously or intermittently changing kinds and concentrations of extracellular matrix components, kinds and concentrations of nutrient components, or kinds and concentrations of components to be added, or culture conditions such as pH and temperature. Thus, it is possible to establish an extracellular matrix environment more similar to the living body in a culture apparatus. Further, it is also possible to regenerate a tissue having a certain inclined structure such as the intestine or ureter by loading a plurality of cellular species (e.g., smooth muscle cells and vascular endothelial cells), in addition to the cell adhesion substrate, simultaneously or at an appropriate time interval, into the closed circulation type culture apparatus.

Further, it is also possible to collect a laminated high-density cultured tissue produced by this method and continuously culture the tissue in a non-circulating culture fluid with the same or different formulation containing an extracellular matrix component and one or more kinds of animal cells.

Thus, according to the apparatus of the present invention, a uniform high-density cultured tissue can be quickly and surely formed while quickly and surely forming a high-density cultured tissue in which a plurality of structures are integrated or combined. Examples of such high-density cultured tissue include the tissues of the respective parts of the human body, such as the skin, cartilage, blood vessel, nerve, ureter, heart, liver, skeletal muscle or internal organs, and tumor tissues.

### EXAMPLES

Hereinafter, the present invention is described in more detail with reference to examples. The present invention is not limited by these examples in any way.

[Preparation of EGF-CBD]

(1) A region of base Nos. 2719 to 3391 in DNA SEQ ID NO: 1 of Clostridium histolyticum colH (GenBank Accession No. D29981) was inserted into the SmaI site of a pGEX-4T-2 plasmid by an ordinary method.

(2) DNA (SEQ ID NO: 6) formed of a base sequence of base Nos. 3308 to 3448 in cDNA SEQ ID NO: 4 of preproEGF of Rattus norvegicus (GenBank Accession No. U04842) was amplified by a PCR method so as to have a BamHI site at the 5′-end and one nucleotide (G residue) for alignment of a reading frame of a fusion protein and an EcoRI site at the 3′-end. The fragment was inserted into the BamHI-EcoRI site of the expression vector according to the item (1) by an ordinary method. The resulting expression plasmid has a reading frame (SEQ ID NO: 7) that encodes a GST-EGF-CBD fusion protein (SEQ ID NO: 8).

(3) As the expression vector for prokaryotic cells was used, the obtained expression plasmid according to the item (2) was introduced into Escherichia coli (Escherichia coli BL21 Codon Plus RIL) by an electroporation method.

In a 2-L flask, 500 mL of a 2×YT-G medium were placed. Then, a liquid medium was prepared by addition of 0.5 mL of a 50 mg/mL ampicillin aqueous solution. To the medium, 10 mL of a preculture medium (a transformant of Escherichia coli BL21 was cultured overnight in 50 mL of the same medium) were inoculated. Then, the culture fluid was subjected to shaking culture at 37° C. until the turbidity (O. D.₆₀₀) of the culture fluid reached about 0.7. Here, 5 mL of a 0.1 M isopropyl-β-D-thiogalactopyranoside (IPTG) aqueous solution were added to the culture fluid and cultured at 37° C. for 2 hours. After that, 5 mL of a 0.1 M phenylmethylsulfonyl fluoride (PMSF) isopropanol solution were added and the culture fluid was then centrifuged at 6,000×g at 4° C. for 10 minutes to collect a transformant. Bacterial cells were suspended in 7.5 mL of a phosphate buffered saline (PBS) containing 1 mM PMSF and subjected to cell breakage treatment with a French press. A 20% Triton X-100 solution was added to the suspension at a volume of 1/19 of the suspension and the mixture was stirred at 4° C. for 30 minutes. The lysate was centrifuged at 15,000×g at 4° C. for 30 minutes and the resulting supernatant was then centrifuged again under the same condition. The supernatant was provided as a cleared lysate solution. To glutathione-sepharose beads (2 mL), the cleared lysate solution was added and stirred at 4° C. for 1 hour to bind a GST-EGF-CBD fusion protein to the beads. After washing the beads with 12 mL of PBS five times, the beads were suspended in a small amount of PBS and loaded onto a column. The fusion protein was eluted with 50 mM Tris-HCl (pH 8.0) and 10 mM glutathione solution. Five units of thrombin per mg of the fusion protein were added and the mixture was subjected to a reaction at 25° C. for 10 hours to cleave a GST tag. After that, dialysis against 300 mL of PBS at 4° C. for 12 hours was repeated four times. The dialyzed cleavage product was added to a column loaded with fresh glutathione-sepharose beads (2 mL) washed with PBS and directly eluted. As a result, the GST tag was removed and EGF-CBD (SEQ ID NO: 8; 225 to 491) without the GST tag was obtained.

### Example 1

### Production of Artificial Skin

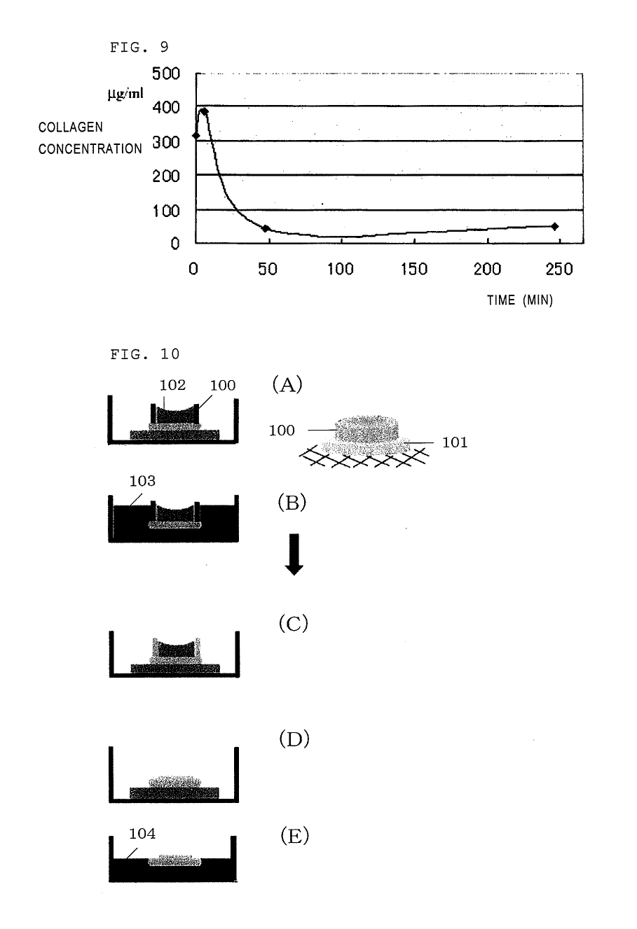

Type I atelocollagen (I-AC; KOKEN Co., Ltd.) extracted from bovine skin and human fibroblasts (HFO; 2×10⁷ cells) were circulated in a reactor for 6 hours. As a result, about 1 g of an artificial connective tissue in terms of wet weight was able to be obtained. The concentration of type I collagen contained in the circulating culture fluid in the closed circulation circuit of the reactor was measured over time. As a result, the concentration of type I atelocollagen in the culture fluid was quickly decreased to about 1/10 after 50 minutes of the onset of circulation (FIG. 9). Thus, dissolved type I collagen in the culture fluid was considered to be accumulated in the reactor as a result of formation of collagen microfibrils by polymerization.

It should be noted that the following reactor was used in this example.

[Reactor]

The reactor has a cylindrical shape of 22 mm in diameter and 17 mm in height (FIG. 1A). In the reactor, a metal spacer (11), a silicon rubber ring (12), a PLA sheet (13), a stainless-steel mesh (14), and a silicon rubber ring (15) are placed in the stated order from top to bottom on a rib (flange) (18) protruded inwardly in a stainless-steel cylinder (16) having slits (17) (FIG. 1B). The extracellular matrix and the cells in the culture fluid are deposited on the PLA sheet (FIG. 1C). In FIG. 1A and FIG. 10, each arrow indicates the direction of a circulating solution. FIG. 1B shows an inner structure of the reactor. As shown in FIG. 10, a high-strength artificial tissue (10) is deposited on the PLA sheet.

A connective tissue prepared using the above-mentioned reactor was transferred to a culture fluid supplemented with an inhibitor (CGS; 10 mM/mL) for a matrix metalloprotease having a tissue disruptive action and ascorbic acid 2-glucopyranose (AA2G; 84.3 mg/mL) as a vitamin C derivative, and a glass cylinder of 10.5 mm in inner diameter and 5 mm in height was then placed (FIG. 10). FIG. 10 shows a method of seeding epidermal cells. A glass cylinder (glass ring) (100) is settled on an artificial dermis (101) taken out from the reactor. The culture fluid (0.4 mL) (102) obtained by adding previously prepared EGF-CBD and human epidermal cells (hEK) (4×10⁵/400 μL) and suspending the mixture was filled in the inside of the glass ring (FIG. 10A). About 3 mL of a medium for skin model (103) were loaded in the outside of the glass ring (FIG. 10B) and the whole was then placed in a CO₂ incubator at 37° C. and left to stand still for 24 hours. After 24 hours, the medium in the inside and outside of the glass ring is removed by suction (FIG. 10C). Then, the glass ring is removed with tweezers so that a hEK layer remains on the gel (FIG. 10D). Next, a medium for skin model (104) is loaded so as to immerse the gel and a gas/liquid culture is then started (FIG. 10E). Thus, an artificial skin can be obtained within 2 weeks.

The thus prepared artificial skin was searched by optical microscopy. As a result, an epidermal layer was observed on an artificial dermal layer formed of fibroblasts and collagen microfibrils. The upper layer portion thereof was keratinized (FIG. 5). FIG. 5 shows an optical microscope image (hematoxylin-eosin staining) of the artificial skin prepared using the reactor. The artificial layer is formed of three layers of the epidermis (E), dermis (D), and a support in descending order. The epidermal layer is formed of three to five layers of epidermal cells laminated to each other. The uppermost layer tends to be keratinized. In the dermal layer, fibroblasts having many projections are present in gaps between collagen fibers. In the lowermost layer, fibers of the support can be observed (100 μm scale).

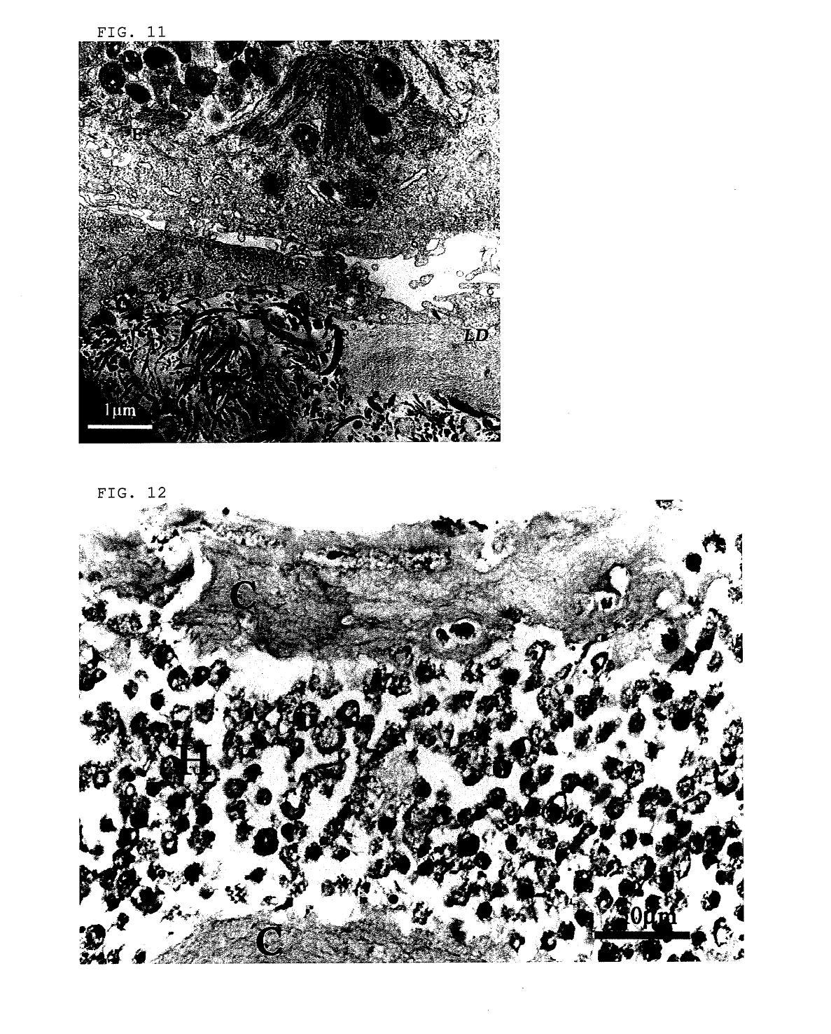

In addition, desmosomes were also formed on the echinate layer, the number of which is small as compared to that in the normal skin (FIG. 11). FIG. 11 shows an electron microscope image of the artificial skin prepared using the reactor. In the epidermal cells (E), many keratin fibers (K), mitochondria, and lysosomes are observed. In the dermis (D), many collagen microfibrils are present in a complicated manner. The basal membrane (LD) is intermittently formed on the boundary between the dermis and the basal epidermal cells (1 μm scale).

### Example 2

### Production of Artificial Liver

The capsule of liver is a connective tissue in which fibroblasts and collagen microfibrils are accumulated at a high density, and is a tissue complex having hepatic cell cords, sinusoids, Glisson's capsule, and the like produced by hepatic parenchymal cells, which are arranged in three dimensions in the capsule. Thus, the reconstitution of a hepatic tissue having the capsule with properties of the connective tissue was attempted. A bioreactor (manufactured by ABLE Corporation) was used as a reactor. Then, a PET mesh sheet was used as a support, and 100 mL of DMEM containing 0.5 mg/mL type I atelocollagen supplemented with fibroblasts (HFO; 1.0×10⁷ cells) were circulated for 6 hours. Subsequently, the circulating solution was replaced with 50 mL of DMEM. Then, just after onset of circulation, a solution prepared by suspending HepG2 cells (2 to 4×10⁷ cells) in 2 mL of DMEM was loaded into a circuit from the upstream of the reactor over 5 to 10 minutes and circulated for additional 2 hours. Subsequently, 50 mL of DMEM containing 0.5 mg/mL type I atelocollagen were circulated for 3 hours to prepare a laminated artificial hepatic tissue. The laminated hepatic tissue was transferred to a circulation culture type reactor and subjected to circulation culture for additional 3 days.

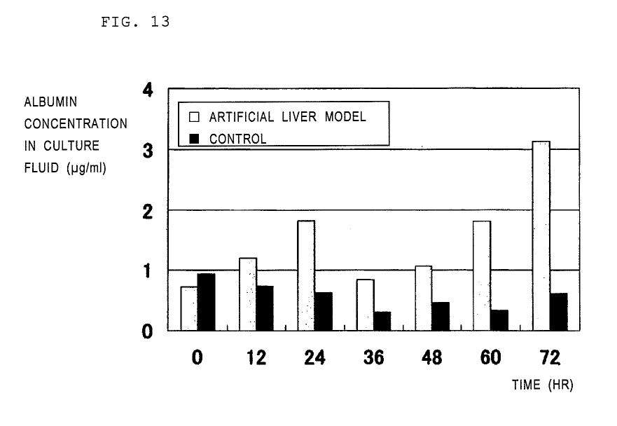

After the closed circulation culture for 11 hours in total, a while jelly-like tissue mass formed of collagen microfibrils, fibroblasts, and HepG2 cells was deposited on the PET sheet. As a result of optical microscope observation, the HepG2 cells were accumulated between connective tissues of two layers formed of collagen microfibrils and fibroblasts (FIG. 12). FIG. 12 shows an optical microscope image (hematoxylin-eosin staining) of the artificial liver prepared using the reactor. Many hepatic cells (HepG2; H) are observed between upper and lower layers of artificial connective tissues (C) (50 μm scale).

It was observed that an albumin synthesizing ability of the thus prepared artificial liver was several times as high as that of the HepG2 cells subjected to mixed culture with fibroblasts (HFO) on a plastic dish (FIG. 13). FIG. 13 shows a time-dependent change in albumin concentration in the culture fluid. To evaluate the albumin producing ability of a three-dimensional complex hepatic tissue, a comparison with the results obtained when fibroblasts (HFO) and hepatic cells (HepG2) were mixed and cultured on a plastic dish was performed. When the concentration of albumin in the culture fluid was quantitatively assayed, on day 3 of the culture, the three-dimensional complex hepatic tissue showed as high a value as 4 to 5 times as compared to the case of a plate culture.

[Albumin Synthesizing Ability of Artificial Liver]

Albumin is synthesized in the liver and secreted in blood. The whole body cells incorporate and utilize albumin from blood. The normal serum concentration of albumin falls within the range of 3.8 to 5.3 g/dL (38,000 to 53,000 μg/mL). Therefore, the functions of the prepared artificial liver can be evaluated by investigating the albumin synthesizing ability. In this example, HepG2 cells established from neoplastic hepatic cells are used instead of hepatic cells. Therefore, the albumin synthesizing ability originally shows a low value. Then, an albumin concentration in the culture fluid was measured by a competitive ELISA method using an ALBUWELL II assay kit (Exowell Inc.). The concentration of albumin secreted into the culture fluid on day 3 of culture was about 0.5 μg/mL in the normal plate culture, whereas showed as high a value as 3 μg/mL or more in the case of making a three-dimensional complex tissue using the method of the present invention (FIG. 13).

### INDUSTRIAL APPLICABILITY

According to the present invention, the three-dimensional cultured artificial tissue, which cannot be obtained by a culture method on a culture dish and is hardly obtained by a method including attaching a cell sheet, can be easily produced. A high-strength complex artificial tissue can be prepared according to the method of the present invention as long as the user has elementary knowledge and skills with respect to cell culture. Thus, an artificial tissue of interest can be easily produced in a medical field that requires a tissue for transplantation or a research institute that requires an artificial tissue in clinical trials for new drugs and the like.

### DESCRIPTION OF REFERENCE NUMERALS

10 high-strength artificial tissue
11 spacer
12 silicon rubber ring
13 PLA sheet
14 stainless-steel mesh
15 silicon rubber ring
16 stainless-steel cylinder
17 slit
18 rib (flange)
100 glass ring
101 artificial dermis
102 culture fluid
103, 104 medium for skin model

### FIG. 2

(1) EPITHELIAL TISSUE
(2) CONNECTIVE TISSUE
(3) SMOOTH MUSCLE TISSUE
(4) PLA SHEET

### FIG. 3

(1) AIR
(2) EPIDERMAL CELL LAYER
(3) CULTURE FLUID
(4) METAL MESH
(5) ARTIFICIAL DERMIS

### FIG. 4

(1) EPIDERMAL LAYER
(2) DERMAL LAYER

### FIG. 6

(1) EPIDERMAL LAYER
(2) DERMAL LAYER

### FIG. 7

(1) ITO CELL
(2) SINUSOID
(3) ENDOTHELIAL CELL
(4) DISSE CAVITY
(5) HEPATIC CELL
(6) FIBROBLAST
(7) CAPSULE

### FIG. 8

(1) COLLAGEN MICROFIBRIL
(2) FIBROBLAST
(3) HEPATIC CELL
(4) POLYLACTIC ACID SHEET

### FIG. 9

(1) COLLAGEN CONCENTRATION
(2) TIME (MIN)

### FIG. 13

(1) ALBUMIN CONCENTRATION IN CULTURE FLUID
(2) TIME
(3) ARTIFICIAL LIVER MODEL
(4) CONTROL

1.-9. (canceled)

10. A method of producing an artificial tissue, including culturing one or more kinds of animal cells in a cell culture fluid containing a collagen-binding cell growth factor and collagen microfibrils.

11. The method of producing an artificial tissue according to claim 10, including producing a laminated high-density cultured artificial tissue by laminating high-density collagen microfibrils in which the one or more kinds of animal cells are embedded, including the steps of: providing a liquid flow-controlling member (such as a poly lactic acid sheet) and a mesh member in contact with or close to each other in a flow path, in which a cell culture fluid containing one or more kinds of animal cells and collagen microfibrils is subjected to circulation culture, so that the mesh member is located on the back surface of the liquid flow-controlling member with respect to a liquid flow; producing a high-density cultured tissue by accumulating the collagen microfibrils and animal cells at a high density on the surface of the liquid flow-controlling member; and subsequently performing at least once an operation of forming a different high-density cultured tissue on the tissue using a different cell culture fluid containing collagen microfibrils and one or more kinds of animal cells, thereby forming a laminated high-density cultured tissue, in which the method includes incorporating a collagen-binding cell growth factor into a circulating culture fluid in at least one step of producing a high-density cultured tissue out of the first and subsequent steps of producing a high-density cultured tissue.

12. The method of producing an artificial tissue according to claim 10, in which the cell growth factor of the collagen-binding cell growth factor is one or two or more selected from the group consisting of an epidermal growth factor (EGF), a fibroblast growth factor (FGF), a platelet derived growth factor (PDGF), a hepatocyte growth factor (HGF), a transforming growth factor (TGF), a neurotrophic factor (NGF), a vascular endothelial growth factor (VEGF), and an insulin-like growth factor (IGF).

13. The method of producing an artificial tissue according to claim 10, further including reconstructing an artificial skin using a collagen-binding epidermal growth factor (EGF-CBD) as the collagen-binding cell growth factor in combination with an epidermal cell.

14. The method of producing an artificial tissue according to claim 13, in which the reconstructing of the artificial skin includes: providing a liquid flow-controlling member and a mesh member in contact with or close to each other in a flow path, in which a cell culture fluid containing collagen microfibrils and one or more kinds of animal cells is subjected to circulation culture, so that the mesh member is located on the back surface of the liquid flow-controlling member with respect to a liquid flow; producing a high-density dermis-like tissue through a closed circulation type high-density tissue culturing step including producing a high-density culture tissue by accumulating the collagen microfibrils and animal cells at a high density on the surface of the liquid flow-controlling member; and subsequently reconstructing an artificial skin using a collagen-binding epidermal growth factor (EGF-CBD) in combination with an epidermal cell.

15. The method of producing an artificial tissue according to claim 10, further including reconstructing a tubular organ.

16. A method of producing an artificial tissue, including the steps: providing a liquid flow-controlling member and a mesh member in contact with or close to each other, in a flow path in which a cell culture fluid containing collagen microfibrils and one or more kinds of animal cells is subjected to circulation culture, so that the mesh member is located on the back surface of the liquid flow-controlling member in relation to a liquid flow; producing a high-density cultured tissue by accumulating the collagen microfibrils and animal cells at a high density on the surface of the liquid flow-controlling member; and subsequently performing at least once an operation of forming a different high-density cultured tissue on the above-mentioned tissue using a different cell culture fluid containing collagen microfibrils and one or more kinds of animal cells, thereby forming a laminated high-density cultured artificial tissue, in which the method includes:
(1) producing a connective tissue corresponding to the capsule of the liver;
(2) laminating a hepatic cell layer regarded as a hepatic cell on the connective tissue; and
(3) producing a layer regarded as a connective tissue in the liver to reconstruct an artificial liver.

17. The method of producing an artificial tissue according to claim 11, in which the liquid flow-controlling member is a biodegradable sheet.

18. An artificial tissue, which is produced by the method according to claim 10.
